(19) United States
(12) Patent Application Publication
(10) Pub. No.: US 2024/0065772 A1
(43) Pub. Date: Feb. 29, 2024

(54) NAVIGATION ASSISTANCE IN A MEDICAL PROCEDURE

(71) Applicant: Cordiguide Ltd., Tel Aviv (IL)

(72) Inventors: Yoni Levi, Haifa (IL); Rafael Beyar, Haifa (IL); Yehoshua Y Zeevi, Haifa (IL)

(73) Assignee: Cordiguide Ltd., Tel Aviv (IL)

(21) Appl. No.: 18/262,182

(22) PCT Filed: Feb. 2, 2022
(86) PCT No.: PCT/IB2022/050879

§ 371 Date: Jul. 19, 2023

Related U.S. Application Data
(60) Provisional application No. 63/199,935, filed on Feb. 3, 2021.

(51) Int. Cl. A61B 34/20; A61B 34/10; A61B 90/00; G06T 7/00

(52) U.S. Cl. CPC A61B 34/20; A61B 34/10; A61B 90/37; G06T 7/0014; A61B 2034/107; A61B 2090/376; G06T 2207/20081; G06T 2207/30101

(57) ABSTRACT

A method for navigation assistance in a medical procedures, the method may include (i) obtaining evaluated images that capture the OOI and the background; wherein the evaluated images are acquired at other points of time during which the one or more injection agents do not flow through at the least one of the BVSs; (ii) determining evaluated image features of the evaluated images by the machine learning process trained to extract the features; (iii) generating predicted BVSs maps for the evaluated images, based on the reference BVSs map information; and (iv) responding to the generating of the predicted BVSs maps and the dynamic movement of the OOI.

### BACKGROUND

Percutaneous Coronary interventions (PCI) include several steps that are all done manually under x-ray fluoroscopy. The steps include Guide Catheter (GC) insertion into the origin of the coronary artery (Ostium), wire navigation along the artery and across the lesion/stenosis to be treated, driving balloons, stents and other devices to the treatment site under various conditions. The set of steps include:

Insertion of the Guide Catheter to the ostium of the treated territory (right or left coronary artery). Guiding catheter positioning, keeping it stable and making sure that it remains at the ostium of the treated arterial system (right or left).

Wire navigation along the arterial pathway leading to the lesion site, crossing the lesion with the wire and advancing to the distal vessel parking position.

Following a successful crossing of the lesion a balloon is advanced to the lesion, with subsequent inflation to expand the lesion and deploy the stent.

Following appropriate stent selection (Diameter and length) it is positioned to cover the entire treated segment of the artery and expand to dilate the artery and remain adjacent to the arterial wall.

Several other methodologies can be used for imaging diagnosis and treatment such as intravascular ultrasound (IVUS), optical coherence tomography (OCT), laser catheter ablation, atherectomy technology etc.

Working along these principles requires training and expertise. While expert physicians can pay attention to every detail simultaneously, less expert operators sometimes fail to see the overall landscape of the intervention leading to non-optimal treatment and errors.

In the case of the catheter based interventional procedures, the distal end of the GC marks the entrance into the intravascular arterial tree, or, later during the procedure, the tip of the GW should be tracked with respect to the dynamic moving background and the BVS. The visual feedback available to the cardiologist/operator about the roadmap of the coronary arteries along this roadmap, is obtained by means of the tissue-and-inserted-devices X-ray shadow, projected onto the monitoring screen, positioned in front of the cardiologist and his assisting team.

To enhance the visibility of the GC, GW, stent, other devices and the arterial roadmap, especially at critical junctions of arterial bifurcations, once the GW has arrived at a roadblock or obstacle creating the lesion, the cardiologist injects a contrast agent. However the arterial tree clearly demonstrated during injections fades away in a few seconds as it washes out.

Contrast injections are required several times during wire and device navigation and positioning throughout the procedure in order to help in the navigation, diagnosis, or treatment of the diseased location. The contrast agent used in medical procedures is harmful to the kidney and the amount used during a medical procedure has to be minimized.

Likewise, to minimize the effect of ionizing radiation on both the patient and the cardiological team, the amount of X-ray has to be reduced to as-a-low-level-as-possible. These safety measures result in a severely compromised image quality and frequent fading of the GC and GW and their trajectory that their continuous display is crucial for the real-time planning and execution of the interventional procedure and in the deployment of the balloon and subsequent positioning of the stent, whenever the treatment of the lesion calls for such a permanent deployment.

Any compromise in the continuity and quality of the projected image may result in failure of the ease of wire navigation precision of deployment of the stent. As a reminder, once the stent placement is completed and the stent is positioned, additional stents are often needed for correction of a suboptimal result resulting in an undesired effect that may have long term adverse clinical consequences such as restenosis.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 illustrates an example of a method;

FIG. 2 illustrates an example of a method;

FIG. 3 illustrates an example of a method;

FIG. 4 illustrates an example of a computerized system;

FIGS. 5, 7, 9 and 11 are examples of evaluated images; and

FIGS. 6, 8, 10 and 12 are examples of overlaid images.

### DETAILED DESCRIPTION OF THE DRAWINGS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that can be executed by the system and should be applied mutatis mutandis to a non-transitory computer readable medium that stores instructions that once executed by a computer result in the execution of the method.

The specification and/or drawings may refer to a computerized system. The computerized system may include a processor—for example a processing circuitry. The processing circuitry may be implemented as a central processing unit (CPU), and/or one or more other integrated circuits such as application-specific integrated circuits (ASICs), field programmable gate arrays (FPGAs), full-custom integrated circuits, etc., or a combination of such integrated circuits. The computerized system may be one or more servers, one or more laptop computers, one or more wearable computers, one or more smartphones, one or more integrated circuits, and the like.

Any combination of any steps of any method illustrated in the specification and/or drawings may be provided.

Any combination of any subject matter of any of claims may be provided.

Any combinations of systems, units, components, processors, sensors, illustrated in the specification and/or drawings may be provided.

Some of the following text may refer to a Percutaneous Coronary interventions (PCI) and to medical elements such as a catheter, a guidewire—but the suggested solution is applicable, mutatis mutandis to any other medical procedures or medical device used.

The suggested solution obtains information from reference images that capture blood vessel segments (BVSs) in which one or more contrast agents flow, and uses the information to provide BVSs map information in relation to evaluated images in which there is no visible contrast agent. This allows a cardiologist/operator to navigate medical elements through the BVSs with less injections—which is safer and reduces the amount of injection material required to fulfill the medical procedure.

Images (reference images and/or evaluated images) may be still images or video segments.

The term overlay means displaying a first information unit over a second information unit. For example—a predicted BVSs map is overlayed over pixels of an evaluated image.

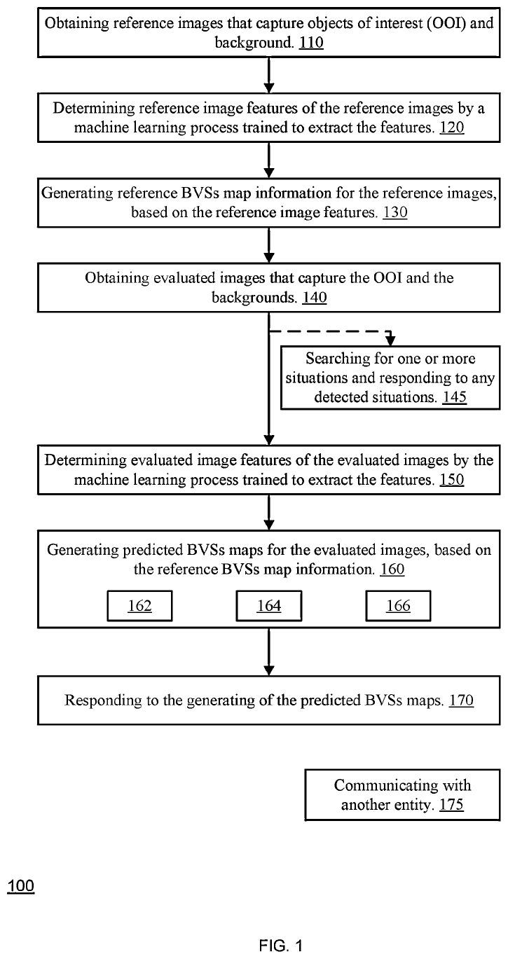

FIG. 1 illustrates an example of method 100 for navigation assistance in a medical procedure.

Method 100 may start by step 110 of obtaining reference images that capture objects of interest (OOI) and background.

The OOI may include blood vessel segments (BVSs).

The OOI may also include other elements (other than BVSs)—for example at least one medial element. The at least one medial element may be inserted in at least some of the BVSs.

The at least one medical element may be selected based on the medical procedure that is related to the execution of method 100.

For example—the at least one medical element may include at least some out of a catheter, one or more guidewires, a balloon, a stent, and the like.

What amounts to an OOI (except a BVS) may be defined in any manner—by a human, by analyzing test images, and the like.

The OOI may be selected based on the medical procedure that is being monitored.

The reference images may be acquired (i) at different points of time, and (ii) while one or more injection agents flow through at least one of the BVSs.

It should be noted that a reference image and an additional reference image may be acquired at the same point of time. The same applied to an evaluated image. For simplicity of explanation the following text will refer to images acquired at different points in time.

Step 110 may be followed by step 120 of determining reference image features of the reference images by a machine learning process that was trained to extract the features.

There may be any combination of reference image features and/or any number of reference image features. The same is applicable, mutatis mutandis, to evaluated image features.

Non-limiting examples of image features include at least some out of:
a. A classification feature. The classification feature may classify any image element (pixel or a set of pixels) to multiple classes such as background or OOI. The classification may be made to different classes of OOI—for example to a BVS, to a medical element, and the like.
b. An OOI centerline feature. Image elements at the center of the BVS.
c. A BVS orientation feature.
d. OOI to background distance feature. Any distance may be calculated and in any manner. The OOI to background distance of a pixel may be, for example, a distance to a closest background pixel. For example—the distance decreases when moving from a centerline pixel of an OOI towards a boundary of the OOI.
e. Blood vessel bifurcation feature.
f. A texture feature.

Any feature related to the shape and/or size and/or location and/or orientation and/or texture of any OOI may be provided.

The machine learning process may be trained (i) using a supervised process, or (ii) using a non-supervised process, or (iii) using a combination of a supervised process and an un-supervised process. For example—the machine learning process may be trained using self-learning training.

The machine learning process may be implemented by one or more neural networks. For example—the machine learning process may be implemented by a neural network having different heads that branch from a representation layer.

That machine learning process may be trained by a self-learning training process enforcing similarity between different views of the same frame. This may include providing different views of a training image to the machine learning process, and enforcing the machine learning process to output substantially the same outputs (for example feature vectors) of the representation layer. The different views may differ from each other by at least one out of noise, translation, rotation, or scale.

Additionally or alternatively, the machine learning process may be trained by a self-learning training process to output from one of the heads of the neural network a reconstructed input image that may be virtually identical to an input image inputted to the neural network.

Step 120 may be followed by step 130 of generating reference BVSs map information for the reference images, based on the reference image features.

The reference BVSs map information may include reference BVSs maps. For example—providing a BVSs map for each reference image.

The patient breathes during the acquisition of the reference images—and may perform additional movements during the acquisition of the reference images. The reference BVSs map information may reflect all types of movements, either of rigid bodies (table, X-RAY source) or non-rigid bodies (breathing, beating heart).

The BVSs map information may be a model that represents the changes in the BVSs maps over time. Any model may be provided.

The BVSs map may be represented in any manner—for example—by a directed acyclic graph (DAG).

For example—centerlines of arteries of a reference image may form an arteries' tree. The ostium of the arteries' tree may be located at the catheter's location. A trajectory from the ostium to each of the branches up to their leaves defines the direction of the blood flow, which can be represented as the DAG.

The method may also include representing an OOI by a model, a graph or another representation that is more compact than the raw pixels of the OOI.

For example—an OOI may be represented by a piecewise linear one-dimensional curve, with variable thickness along the curve.

Using a model and/or a graph provides a compact and accurate representation of the BVSs and/or other OOI—and thus saves memory resources, computational resources (for example—when finding a reference image similar to an evaluated image).

The reference BVSs map may include the raw pixels of a reference image that capture the BVSs. Alternatively—when the reference BVSs maps are represented by models and/or by a tree or by any other manner that is more compact that the raw pixels—a saving in computational and/or memory resources are provided. Furthermore—the raw pixels may be noisy—and using a model and/or tree may be less noisy and more accurate.

Step 130 may be followed by step 140 of obtaining evaluated images that capture the OOI and the background. The evaluated images may be acquired at other points of time during which the one or more injection agents do not flow through at the least one of the BVSs.

Method 100 may distinguish between reference images and evaluated images by the presence or absence of contrast agent in the images. Absence—may mean without any trace to a contrast agent—or with an insignificant trace (insignificant may be defined by the user or by any other manner, insignificant may include an amount that does not allow to establish a BVSs map of an image).

Step 140 may be followed by step 150 of determining evaluated image features of the evaluated images by the machine learning process that was trained to extract the features.

Step 150 may be followed by step 160 of generating predicted BVSs maps for the evaluated images, based on the reference BVSs map information.

Assuming that there is a reference BVSs map per reference image then step 160 may include steps 162 and 164. Step 162 may be followed by step 164.

Step 162 may include selecting, for an evaluated image, a corresponding reference image.

The corresponding reference image may have a background that is similar to the background of the evaluated image. For example—the corresponding reference image may have a background that is the most similar background out of the backgrounds of reference images obtained during step 110.

The similarity can be determined in any manner and using any matric or formula.

The similarity may be determined between one or more background features of the reference images (may be determined during step 120) and one or more background features of the evaluated image (may be determined during step 150).

The machine learning process used during steps 120 and 150 may be implemented by a neural network having different heads for different image features. Different heads branch from a representation layer of the neural network. The similarity may be determined based on outputs of the representation-layer.

Additionally or alternatively, the selecting may be based on a presence of at least one anchor within each one of the corresponding reference images and given evaluated image. In this case, step 162 may include searching for the same appearance of the same one or more anchors in the evaluated image and in the corresponding reference image.

Step 164 may include generating the predicted BVSs map (for the evaluated image) based on a reference BVSs map of the corresponding reference image.

Step 164 may include providing the reference BVSs map of the corresponding reference image as the predicted BVSs map of the evaluated image.

Step 164 may include alignment and/or any other processing.

Step 160 may include step 166 of estimating the locations of one or more medical elements within the predicted BVSs maps.

Step 166 of estimating may link between pixels of the same medical element that represent spaced apart parts of the same medical element. For example—linking spaced apart parts of a guidewire that has only partially seen in an evaluated image.

The estimating may include linking between spaced apart pixels of different medical elements that are related to each other. For example—assuming that a catheter is seen in an evaluated image and only a tip of a guidewire extending from the catheter is seen in the evaluated image. In this case the estimating may include linking between the guidewire tip and the catheter (within one or more BVSs).

Step 160 may be followed by step 170 of responding to the generating of the predicted BVSs maps.

Step 170 may include participating in an overlaying of the predicted BVSs maps on the evaluated images.

The participating may include at least one out of:
a. Generating overlay information for overlaying the predicted BVSs maps.
b. Sending the overlay information to another computerized entity such as a display controller or any entity that is responsible to or participated in the display of an evaluated image overlaid by the predicted BVSs map.
c. Storing the overlay information.
d. Sending an alert to the other computerized entity about the existence of the overlay information.
e. Overlaying the predicted BVSs map on a corresponding evaluated image.

The overlay information may include information about one or more OOI that differ from the BVSs—for example—overlaying one or more medical element—even pixels of the medical elements that do not clearly appear in the evaluated image.

Step 170 may include participating in an overlaying of any OOI on the evaluated images.

Step 170 may include aligning a predicted BVSs map with a corresponding evaluated image. Additionally or alternatively, step 170 may include aligning consecutive BVSs maps to each other.

The aligning may be based on a presence of at least one anchor within each one of the predicted BVSs map and the corresponding evaluated image—and/or based on a presence of at least one anchor within each one of the consecutive predicted BVSs maps.

The aligning may be based on one or more evaluated image features.

The aligning may be based on locations of BVSs junctions.

The aligning may include applying a topology-preserving map for vertices in two consecutive BVSs maps. Once this is done, an edges mapping is given. Finally, for any two corresponding edges, the method may establish point-to-point correspondence according to (normalized) distance from an edge start point.

Step 170 may include providing a visual mark at location of interest in an evaluated image. The location of interest may be provided from a man-machine interface such as a touch screen, a keyboard, a mouse, a voice interface, and the like. The location of interest may be provide from a human (for example a cardiologist/operator, a nurse, and the like) of from a non-human entity.

The visual mark may be defined by the same human (or same non-human entity).

Method 100 may include tracking the location of interest over time to provide the mark at the location of interest in multiple evaluated images acquired at different points in time.

There may be multiple repetitions of steps 140-170 per each repetition of steps 110-130.

Steps 110-130 may be executed by a computerized system while steps 140-170 may be executed by the same computerized system or by another computerized system.

Method 100 may also include step 145 of searching for one or more situations and responding to any detected situation.

Step 145 may be preceded by step 140.

Non-limiting examples of situations may include at least one out of:
a. Abrupt change in location of a guidewire and/or catheter.
b. A guidewire and/or catheter goes out of frame (evaluated image).
c. A guidewire tip changes its shape.
d. A guidewire tip is orthogonal (or substantially orthogonal) to artery side.

Abrupt change in the location of the guidewire and/or catheter—what amount per abrupt (at least a predefined location change rate)—may be defined in any manner—for example by a user. For example, an abrupt change if the catheter was located on the left side of an evaluated image, and in the next evaluated images—the catheter was detected at a displaced position (either too deep in the artery or pushed back). This abrupt change may indicate that something is wrong (most likely, the guidewire or the medical device is stuck).

A guidewire tip changes its shape. A shape change that may be of interest may be defined in any manner. Insignificant changes (for example at least one rigid transformation) may be defined in any manner. This may indicate that the GW is pushed against resistance and may be stuck, in which case, continuing to push it forward might harm the arteries. The finding of this situation may include, for example, searching for the pixels of an evaluated image that is indicative of the tip of the guidewire—pixels located at the “end” of the guidewire. These pixels are indicative of the shape of the tip. The shape may be initially determined by the way the cardiologist/operator set the tip. The initial shape may appear in the first evaluated images in which the tip appears. The initial shape is compared to the tip shape in the following evaluated images—to find a change of shape of interest.

A guidewire tip is orthogonal (or substantially orthogonal) to artery side—in this situation there is a danger the guidewire punches a hole in the artery. This situation may include using artery boundary information from the BVSs map information. In case the frontal part of the guidewire gets close to one side of the artery and the direction of the side is orthogonal (or substantially orthogonal) to the direction of the guidewire tip, an alert may be generated. The direction of the artery side may be defined in various manners—for example a direction of a curve representing the artery at the point where the guidewire tip is found. The direction of the guidewire tip may be defined in any manner—for example by the pixels of the guidewire near the artery border. The tip pixels may be, for example, pixels stretched from most frontal pixels of the guidewire.

The responding to the one or more situations may include sending one or more alerts—audio and/or visual and/or tactile. For example—displaying an alert on the evaluated image with the overlaid predicted BVSs map, generating an audio alarm, and the like.

The intensity and/or type of alerts and/or the frequency of the alerts may be a function of the severity of the situation.

The detecting of the situation can be done in any manner—for example may be a rule based and/or any other non-machine learning based detection, may be a machine learning based detection.

A machine learning based detection may be independent from the machine learning process used to extract the features. Alternatively—one or more predefined situation can be detected based on outputs from the machine learning process used to generate the features.

Method 100 may include, for example, step 175 of communicating with another entity. Step 175 may also include responding to the communicating.

The communicating may include communicating with a human using a man machine interface, and/or communicating may include communicating with one or more computerized systems and/or networks and/or storage systems, and the like. The communication may be uni-directional or bi-directional. Any communication protocol may be used.

The responding may include, for example allowing a human to control a display, marking the location of interest, tracking after the location of interest and the like.

For example—a cardiologist or another operator, may at any time, “freeze” a screen, focus on a specific location of the frame (zoom in), and mark the location of interest. The location of interest may be a part of a BVSs map or not. One or more locations of interest may be marked. More than a single location of interest may define (for example by providing boundary points) a region of interest—for example a stenosis area.

Method 100 may include keeping track of the region of interest and/or the one or more locations of interest, and may display them. This may ease the navigation even further, especially when localizing balloons or stents for treatment, therefore, prevent unnecessary injections.

Step 175 may be a part of step 145 and/or a part of step 170—or not be included in any one of steps 145 and/or step 170.

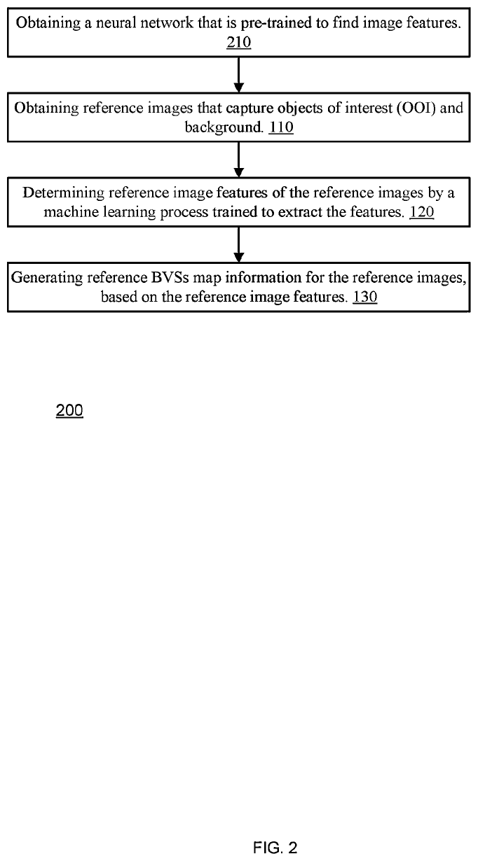

FIG. 2 illustrates method 200 for training a machine learning process.

Method 200 may include step 210 of obtaining a neural network that is pre-trained to find image features—such as the features found in step 120 and/or step 150 of method 100.

The obtaining may include pre-training the machine learning process or receiving a pre-trained machine learning features.

The pre-training may executed on test images of BVSs (and optionally also of one or more OOI) taken from multiple persons. One or more contrast agents should be captured by the test images.

The pre-training of the machine learning process may be a supervised process, or a non-supervised process, or a combination of a supervised process and an un-supervised process. For example—step 210 may include self-learning training.

The machine learning process may be implemented by a neural network having different heads for different image features. The different heads branch from a representation layer of the neural network.

A pre-training of the neural network may be based on one or more outputs of the representation layer. Thus—a cost function may be applied on the one or more outputs of the representation layer.

A pre-training of the neural network may be based on one or more outputs of one or more of the different heads. For example—one or more cost functions may be applied on the outputs of one or more heads of the different heads. For example—a weighted sum of losses from the different heads may be provided as an over cost of the neural network.

A pre-training of the neural network may be based on one or more outputs of one or more of the different heads and also on one or more outputs of the representation layer.

The neural network may of include layers of a u-net type neural network. The last layer of the u-type neural network may be the representation layer.

In case that the different heads branch from different layers of the neural network—the representation layer may be the first layer from which any head branched.

Step 210 may be followed by step 110 of obtaining reference images that capture objects of interest (OOI) and background.

Step 110 may be followed by step 120 of determining reference image features of the reference images by the machine learning process (being pre-trained to extract the features).

Step 120 may be followed by step 130 of generating reference BVSs map information for the reference images, based on the reference image features.

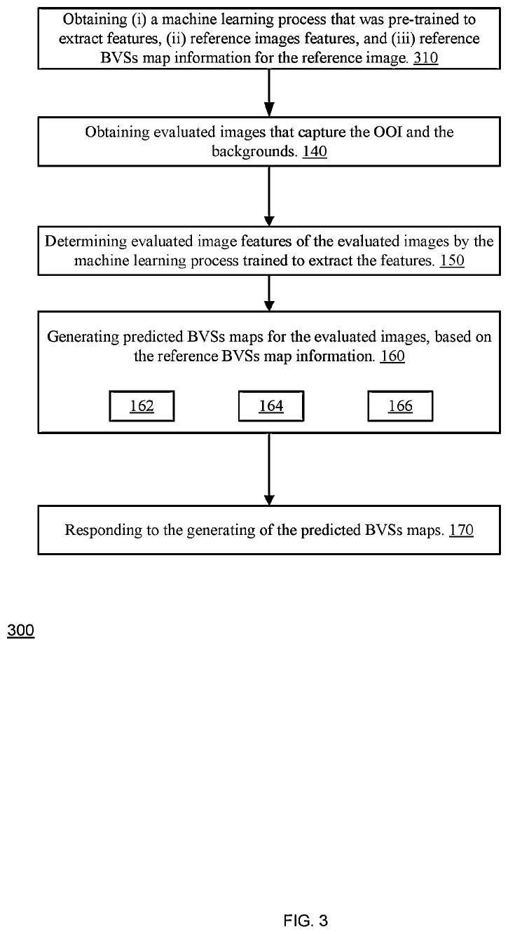

FIG. 3 illustrates method 300 for navigation assistance in a medical procedure.

Method 300 may start by step 310 of obtaining (i) a machine learning process that was pre-trained to extract features, (ii) reference images features, and (iii) reference BVSs map information for the reference images.

Step 310 may be followed by step 140 of obtaining evaluated images that capture the OOI and the background. The evaluated images may be acquired at other points of time during which the one or more injection agents do not flow through at the least one of the BVSs.

Step 140 may be followed by step 150 of determining evaluated image features of the evaluated images by the machine learning process trained to extract the features.

Step 150 may be followed by step 160 of generating predicted BVSs maps for the evaluated images, based on the reference BVSs map information.

Step 160 may be followed by step 170 of responding to the generating of the predicted BVSs maps.

Method 300 may include step 145 and/or step 175.

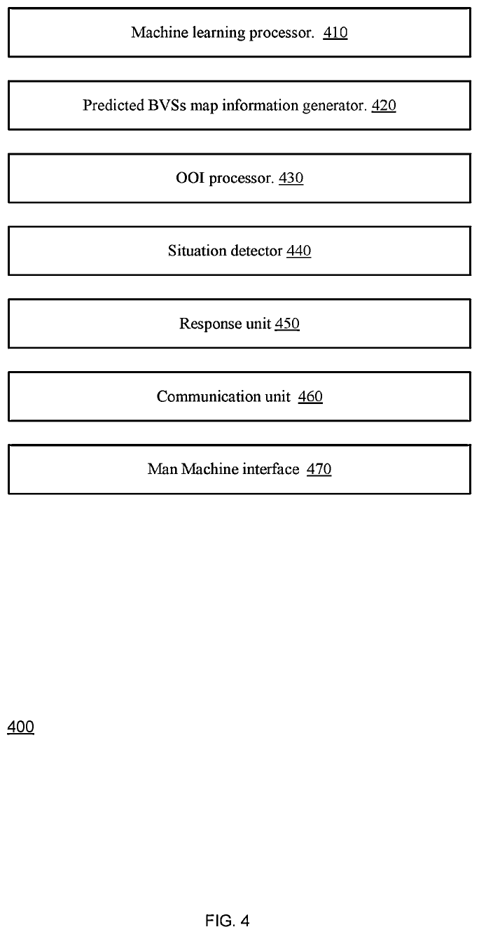

FIG. 4 illustrates an example of a computerized system 400. The computerized system

Computerized system 400 may be configured to execute method 100 and/or method 200 and/or method 300.

Computerized system 400 may include at least some of machine learning processor 410 (may execute, for example steps 120 and 150), predicted BVVs map information generator 420 (may execute, for example, step 160), OOI processor 430 (may participate, for example in the execution of step 160), situation detector 440 (may execute, for example, step 145), response unit 450 (may execute, for example, step 170), communication unit 460 (may execute, for example, step 175), and man machine interface (MMI) 470. Any of the entities may include one or more processing circuits or may executed by or hosted by one or more processing circuits. MMI 470 may be a visual MMI, an audio MMI, a tactile MMI, a mouse, a keyboard, and the like.

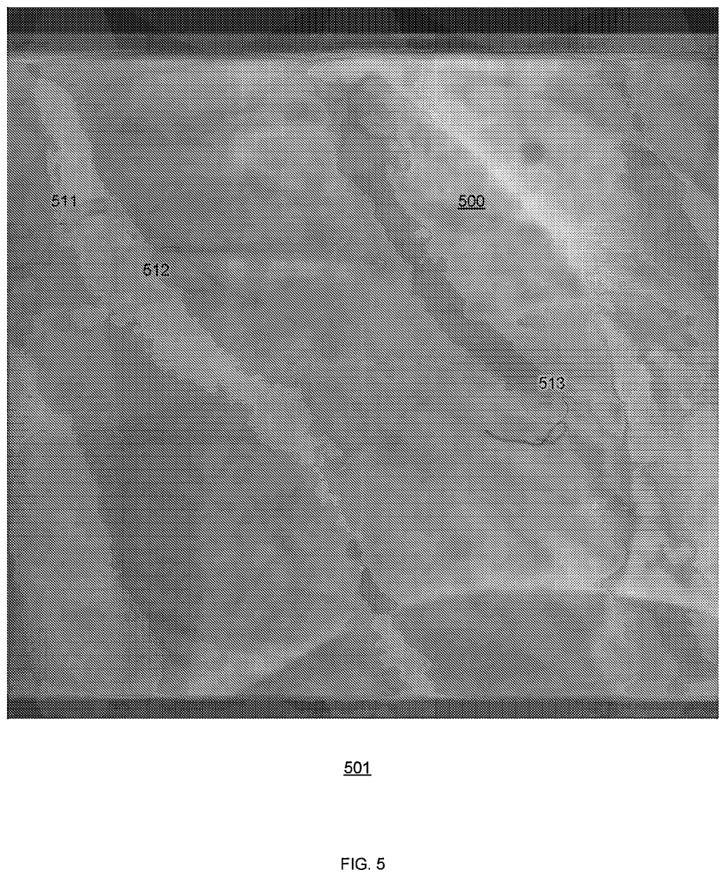

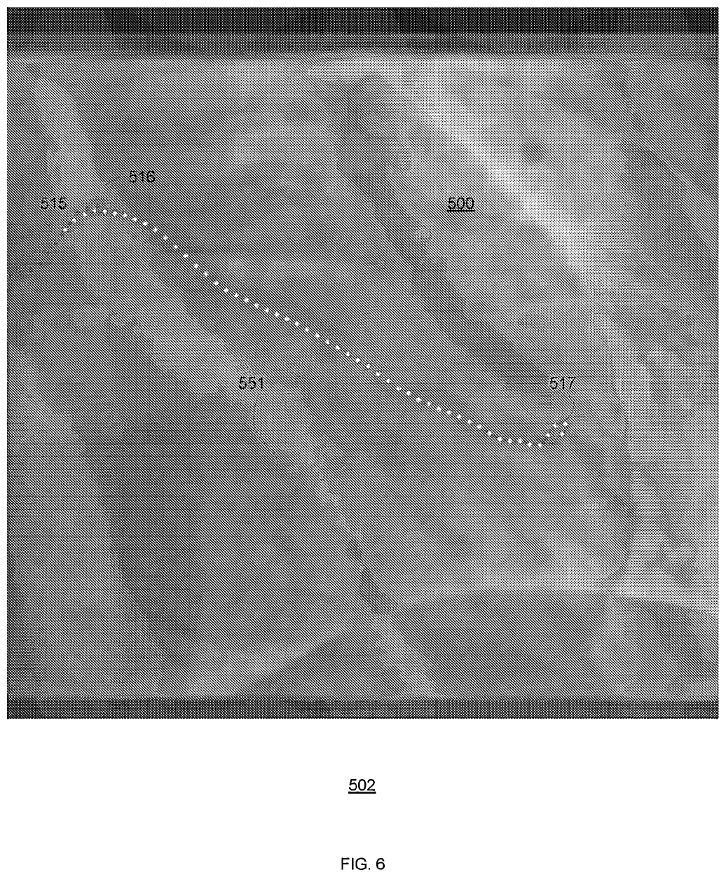

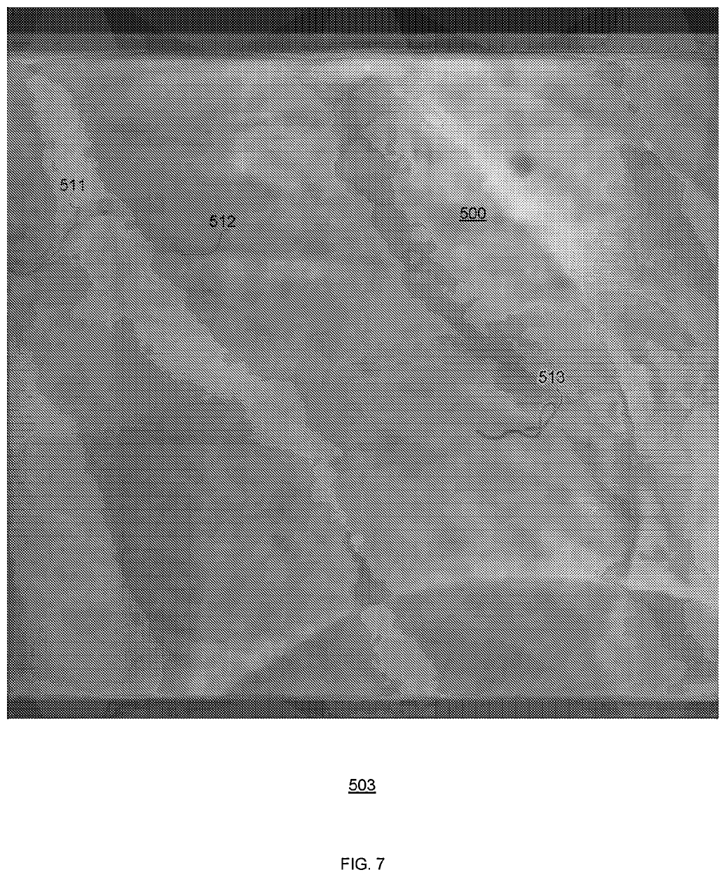

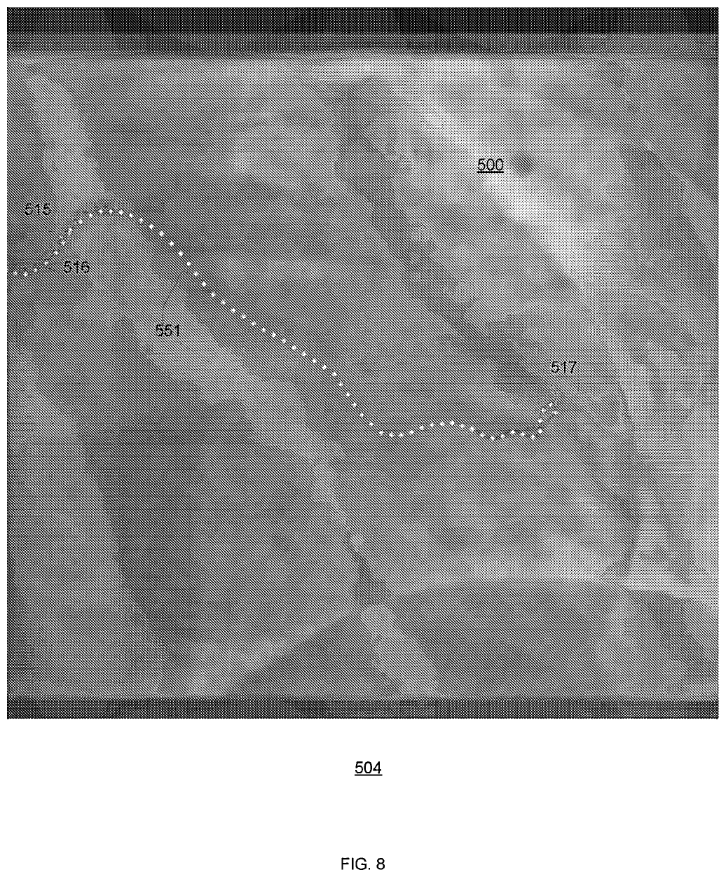

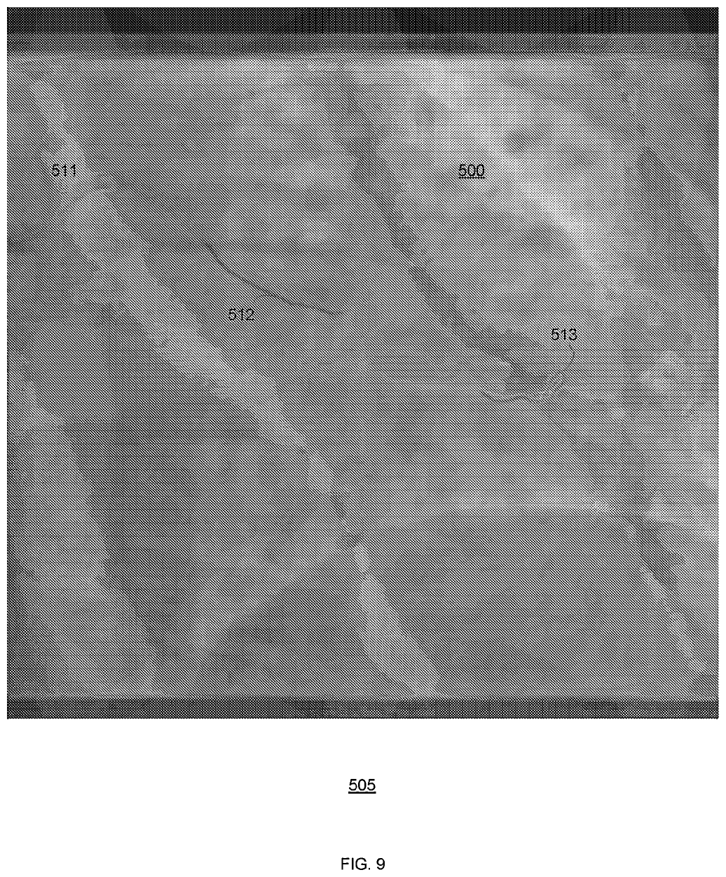

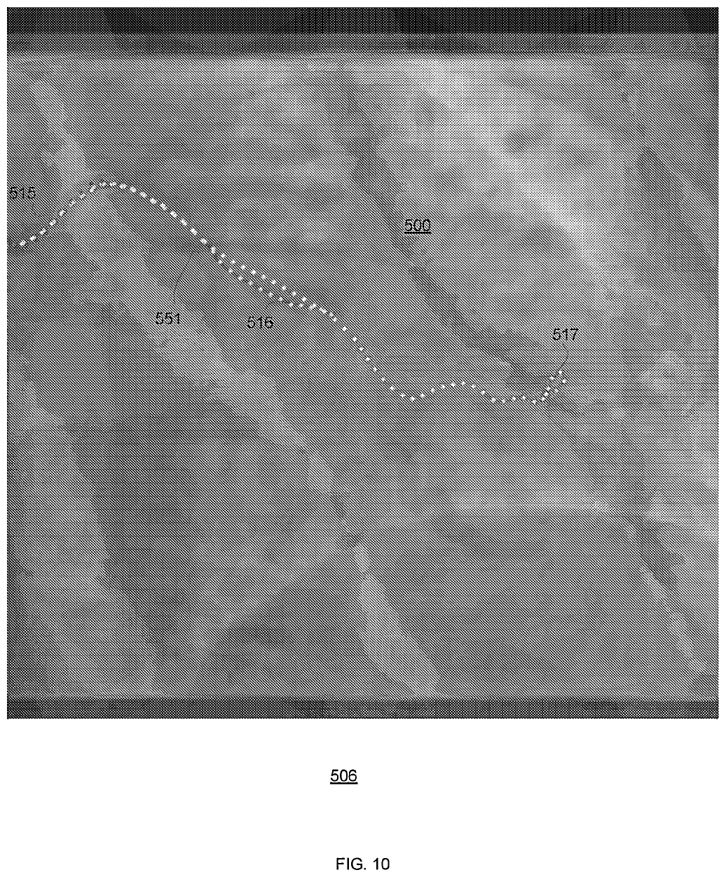

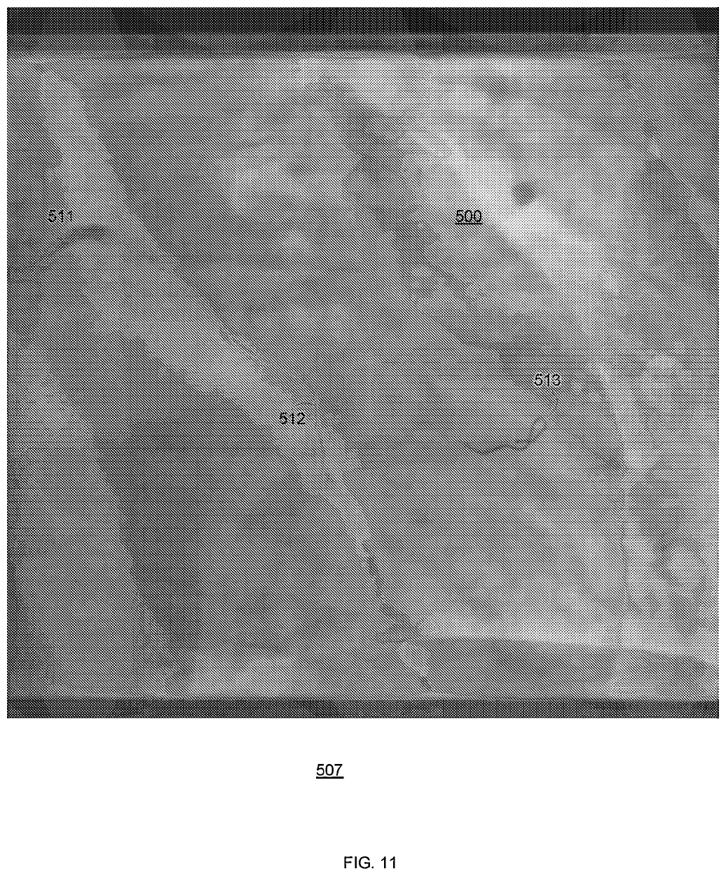

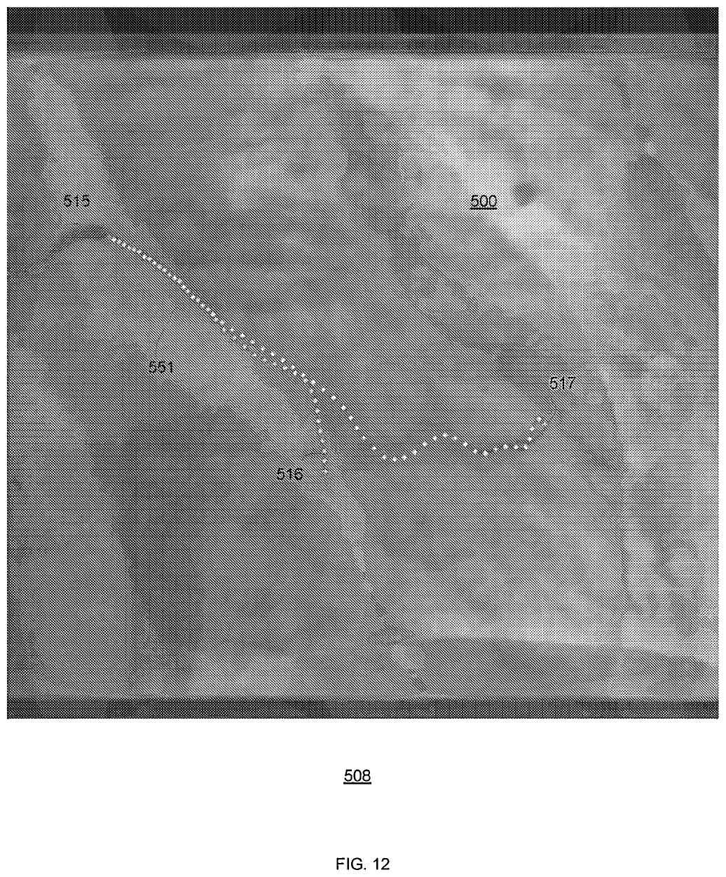

FIGS. 5-12 illustrates example of pairs of image. Each pair includes an evaluated image (for example an image out of images 501, 503, 505 and 507 of FIGS. 5, 7, 9 and 11) and an overlaid image (for example an image out of images 502, 504, 506 and 508 of FIGS. 6, 8, 10 and 12) in which a BVSs map and other OOIs are overlaid. FIGS. 5-12 illustrate different phases of a PCI procedure.

All images show background 500. The evaluated images illustrate one or more parts of a catheter 511, a first guidewire 512, and a second guidewire 513.

At least some of the overlaid images include a predicted BVSs map 551, an estimate 516 of the first guidewire, an estimate 517 of the second guidewire and an estimate 515 of the catheter.

Any mark and/or any visual alert may be provided on any of overlaid images—or elsewhere.

This application provides a significant technical improvement over the prior art—especially an improvement in computer science.

There may be provided a non-transitory computer readable medium for navigation assistance in a medical procedure, the non-transitory computer readable medium may include (a) obtaining reference images that capture objects of interest (OOI) and background; wherein the OOI may include blood vessel segments (BVSs); wherein the reference images are acquired (i) at different points of time, and (ii) while one or more injection agents flow through at least one of the BVSs; (b) determining reference image features of the reference images by a machine learning process trained to extract the features; (c) generating reference BVSs map information for the reference images, based on the reference image features; (d) obtaining evaluated images that capture the OOI and the background; wherein the evaluated images are acquired at other points of time during which the one or more injection agents do not flow through at the least one of the BVSs; (e) determining evaluated image features of the evaluated images by the machine learning process trained to extract the features; (f) generating predicted BVSs maps for the evaluated images, based on the reference BVSs map information; and (g) responding to the generating of the predicted BVSs maps.

The reference BVSs map information may include reference BVSs maps.

The generating of a predicted BVSs map of a given evaluated image may include selecting a corresponding reference image; and generating the predicted BVSs map based on a reference BVSs map of the corresponding reference image.

The selecting may be based on a similarity between a background of corresponding reference image and a background of the given evaluated image.

The evaluated image features and the reference image features that may store instructions for a background feature.

The machine learning process may be implemented by a neural network having different heads for different image features; wherein the different heads branch from a representation layer of the neural network.

The similarity may be determined based on outputs of the representation-layer.

The selecting may be based on a presence of at least one anchor within each one of the corresponding reference image and given evaluated image.

The evaluated image features and the reference image features may include a classification feature, an OOI centerline feature, an a BVS orientation feature.

The evaluated image features and the reference image features further may include a OOI to background distance feature, and a blood vessel junction feature.

The evaluated image features and the reference image features may include a texture feature.

The steps (e) and (f) may be executed one evaluated image at a time.

The machine learning process may be implemented by a neural network having different heads for different image features; wherein the different heads branch from a representation layer of the neural network.

The responding may include participating in an overlaying of the predicted BVSs maps on the evaluated images.

The participating may include overlaying a predicted BVSs map on a corresponding evaluated image.

The participating may include aligning a predicted BVSs map a corresponding evaluated image.

The aligning may be based on a presence of at least one anchor within each one of the predicted BVSs map and the corresponding evaluated image.

The aligning may be based on one or more evaluated image features.

The aligning may be based on locations of BVSs bifurcations.

The responding may include providing a visual mark at location of interest in an evaluated image, wherein the location of interest may be provided from a man-machine interface.

The non-transitory computer readable medium that may store instructions for receiving, from a human, a description of the location of interest.

The responding may include displaying the visual mark at location of interest in an evaluated image, wherein the location of interest may be provided from a man-machine interface.

The OOI may include at least one medical element inserted in at least some of the BVSs.

The at least one medical element may include a catheter and one or more guidewires.

The reference images and the evaluated images may be acquired during a percutaneous coronary intervention (PCI) procedure.

The non-transitory computer readable medium that may store instructions for finding at least one anchor in at least one image out of the reference images and the evaluated images; and responding to the finding.

The machine learning process may be implemented by a neural network having different heads that branch from a representation layer.

The machine learning process may be trained by a self-learning training process enforcing similarity between different views of a same frame.

The self-learning training process may be based on outputs of the representation layer.

The machine learning process may be trained by a self-learning training process to output from one of the heads of the neural network a reconstructed input image that may be virtually identical to an input image inputted to the neural network.

The machine learning process may be trained using a supervised process.

The machine learning process may be trained using a non-supervised process.

The non-transitory computer readable medium may store instructions for detecting a predefined situation and responding to the predefined situation.

Any reference to the term “comprising” or “having” should be applied mutatis mutandis to “consisting” of “essentially consisting of”. For example—a method that comprises certain steps can include additional steps, can be limited to the certain steps or may include additional steps that do not materially affect the basic and novel characteristics of the method—respectively.

The invention may also be implemented in a computer program for running on a computer system, at least including code portions for performing steps of a method according to the invention when run on a programmable apparatus, such as a computer system or enabling a programmable apparatus to perform functions of a device or system according to the invention. The computer program may cause the storage system to allocate disk drives to disk drive groups.

A computer program is a list of instructions such as a particular application program and/or an operating system. The computer program may for instance include one or more of: a subroutine, a function, a procedure, an object method, an object implementation, an executable application, an applet, a servlet, a source code, an object code, a shared library/dynamic load library and/or other sequence of instructions designed for execution on a computer system.

The computer program may be stored internally on a computer program product such as non-transitory computer readable medium. All or some of the computer program may be provided on computer readable media permanently, removably or remotely coupled to an information processing system (also referred to as a computerized system). The computer readable media may include, for example and without limitation, any number of the following: magnetic storage media including disk and tape storage media; optical storage media such as compact disk media (e.g., CD-ROM, CD-R, etc.) and digital video disk storage media; nonvolatile memory storage media including semiconductor-based memory units such as FLASH memory, EEPROM, EPROM, ROM; ferromagnetic digital memories; MRAM; volatile storage media including registers, buffers or caches, main memory, RAM, etc. A computer process typically includes an executing (running) program or portion of a program, current program values and state information, and the resources used by the operating system to manage the execution of the process. An operating system (OS) is the software that manages the sharing of the resources of a computer and provides programmers with an interface used to access those resources. An operating system processes system data and user input, and responds by allocating and managing tasks and internal system resources as a service to users and programs of the system. The computer system may for instance include at least one processing unit, associated memory and a number of input/output (I/O) devices. When executing the computer program, the computer system processes information according to the computer program and produces resultant output information via I/O devices.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

Also for example, the examples, or portions thereof, may implemented as soft or code representations of physical circuitry or of logical representations convertible into physical circuitry, such as in a hardware description language of any appropriate type.

Also, the invention is not limited to physical devices or units implemented in non-programmable hardware but can also be applied in programmable devices or units able to perform the desired device functions by operating in accordance with suitable program code, such as mainframes, minicomputers, servers, workstations, personal computers, notepads, personal digital assistants, electronic games, automotive and other embedded systems, cell phones and various other wireless devices, commonly denoted in this application as ‘computer systems’.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

We claim:

1. A method for navigation assistance in a medical procedure, the method comprises:
a. obtaining reference images that capture objects of interest (OOI) and background; wherein the OOI comprise blood vessel segments (BVSs); wherein the reference images are acquired (i) at different points of time, and (ii) while one or more injection agents flow through at least one of the BVSs;
b. determining reference image features of the reference images by a machine learning process trained to extract the features;
c. generating reference BVSs map information for the reference images, based on the reference image features;
d. obtaining evaluated images that capture the OOI and the background; wherein the evaluated images are acquired at other points of time during which the one or more injection agents do not flow through at the least one of the BVSs;
e. determining evaluated image features of the evaluated images by the machine learning process trained to extract the features;
f. generating predicted BVSs maps for the evaluated images, based on the reference BVSs map information; and
g. responding to the generating of the predicted BVSs maps.

2. The method according to claim 1 wherein the reference BVSs map information comprises reference BVSs maps.

3. The method according to claim 2 wherein a generating of a predicted BVSs map of a given evaluated image comprises: selecting a corresponding reference image; and generating the predicted BVSs map based on a reference BVSs map of the corresponding reference image.

4. The method according to claim 3 wherein the selecting is based on a similarity between a background of corresponding reference image and a background of the given evaluated image.

5. The method according to claim 4 wherein the evaluated image features and the reference image features comprise a background feature.

6. The method according to claim 4 wherein the machine learning process is implemented by a neural network having different heads for different image features; wherein the different heads branch from a representation layer of the neural network.

7. The method according to claim 6 wherein the similarity is determined based on outputs of the representation-layer.

8. The method according to claim 4 wherein the selecting is based on a presence of at least one anchor within each one of the corresponding reference image and given evaluated image.

9. The method according to claim 1 wherein the evaluated image features and the reference image features comprise a classification feature, an OOI centerline feature, an a BVS orientation feature.

10. The method according to claim 9 wherein the evaluated image features and the reference image features further comprise a OOI to background distance feature, and a blood vessel junction feature.

11. The method according to claim 1 wherein the evaluated image features and the reference image features comprise a texture feature.

12. The method according to claim 2 wherein steps (e) and (f) are executed one evaluated image at a time.

13. The method according to claim 12 wherein the machine learning process is implemented by a neural network having different heads for different image features; wherein the different heads branch from a representation layer of the neural network.

14. The method according to claim 1 wherein the responding comprises participating in an overlaying of the predicted BVSs maps on the evaluated images.

15. The method according to claim 14 wherein the participating comprises overlaying a predicted BVSs map on a corresponding evaluated image.

16. The method according to claim 14 wherein the participating comprises aligning a predicted BVSs map a corresponding evaluated image.

17. The method according to claim 16 wherein the aligning is based on a presence of at least one anchor within each one of the predicted BVSs map and the corresponding evaluated image.

18. The method according to claim 16 wherein the aligning is based on one or more evaluated image features.

19. The method according to claim 16 wherein the aligning is based on locations of BVSs bifurcations.

20. The method according to claim 1 wherein the responding comprises providing a visual mark at location of interest in an evaluated image, wherein the location of interest is provided from a man-machine interface.

21. The method according to claim 20 comprising receiving, from a human, a description of the location of interest.

22. The method according to claim 20 wherein the responding comprises displaying the visual mark at location of interest in an evaluated image, wherein the location of interest is provided from a man-machine interface.

23. The method according to claim 1 wherein the OOI comprise at least one medical element inserted in at least some of the BVSs.

24. The method according to claim 23 wherein the at least one medical element comprises a catheter and one or more guidewires.

25. The method according to claim 24 wherein the reference images and the evaluated images are acquired during a percutaneous coronary intervention (PCI) procedure.

26. The method according to claim 1 comprising finding at least one anchor in at least one image out of the reference images and the evaluated images; and responding to the finding.

27. The method according to claim 1 wherein the machine learning process is implemented by a neural network having different heads that branch from a representation layer.

28. The method according to claim 27 wherein the machine learning process is trained by a self-learning training process enforcing similarity between different views of a same frame.

29. The method according to claim 28 wherein the self-learning training process is based on outputs of the representation layer.

30. The method according to claim 27 wherein the machine learning process is trained by a self-learning training process to output from one of the heads of the neural network a reconstructed input image that is virtually identical to an input image inputted to the neural network.

31. The method according to claim 1 wherein the machine learning process is trained using a supervised process.

32. The method according to claim 1 wherein the machine learning process is trained using a non-supervised process.

33. The method according to claim 1 comprising detecting a predefined situation and responding to the predefined situation.

34. A non-transitory computer readable medium that stores instruction for executing a method according to any of the previous claims.

35. A computerized system that is configured to execute a method according to any claim of claims 1-33.
